(19) United States
(12) Patent Application Publication
(10) Pub. No.: US 2026/0297019 A1
(43) Pub. Date: Oct. 1, 2026

(54) METHOD FOR PREPARING ISOCYANATE BY COMBINING SUPERCRITICAL PHOSGENATION METHOD WITH PIPING METHOD

(71) Applicant: MOJIA (SHANGHAI) BIOTECH CO., LTD., Shanghai (CN)

(72) Inventors: Yonghe XUE, Shanghai (CN); Chengliang LU, Shanghai (CN); Man Kit LAU, Shanghai (CN); Haixin YUAN, Shanghai (CN); Jiate LIU, Shanghai (CN); Ansen CHIEW, Shanghai (CN)

(21) Appl. No.: 18/881,298

(22) PCT Filed: Jul. 6, 2022
(86) PCT No.: PCT/CN2022/104181

§ 371 Date: Jan. 5, 2025

(51) Int. Cl. C07C 263/10; C07C 263/20

(52) U.S. Cl. CPC C07C 263/10; C07C 263/20

(57) ABSTRACT

The present application relates to a method for preparing an isocyanate. Specifically, the present application provides a method for preparing an isocyanate by combining a supercritical phosgenation method with a piping method.

### FIELD OF THE INVENTION

The present application relates to a method for preparing an isocyanate, and specifically to a method for preparing an isocyanate by combining a supercritical phosgenation method with a piping method.

### BACKGROUND

Isocyanates are a class of compounds containing one or more isocyanate groups, including aliphatic isocyanates, aromatic isocyanates, unsaturated isocyanates, halogenated isocyanates, thioisocyanates, phosphorus-containing isocyanates, inorganic isocyanates, blocked isocyanates, and so on. Because of the highly unsaturated isocyanate groups contained therein, they are highly chemically active and can undergo important chemical reactions with many substances. Therefore, isocyanates are widely used in polyurethane, polyurethane-urea, polyurea, polymer modification, reagents for organic synthesis, agriculture, medicines and other fields.

In the prior art, the principle of preparing an isocyanate with phosgene and amine has been well known. Due to the high reactivity of amines (especially, aliphatic diamine), amines that have not yet participated in the reaction may react with reaction products and intermediates during the phosgenation reaction, to produce by-products, such as amine hydrochloride, urea, and biuret, etc. To avoid the production of by-products, a capping reagent (such as HCl) can be selected to protect the amino group (—NH₂) of the amine to form an amine salt. However, the reactivity of the amine salt is much lower than that of free amine, and the amine salt is almost insoluble in any common organic solvent and can only be dispersed in the solvent. Practice has proved that the phosgenation reaction with an amine salt as the raw material usually requires the use of a large amount of solvent as a dispersant, and the content of the amine salt in the solvent is often less than 10%, and the residence time of the phosgenation reaction is often several hours or even more than ten hours.

Therefore, there is still a need for an optimized method for preparing an isocyanate.

### BRIEF SUMMARY OF THE INVENTION

The present application aims to provide a method for preparing an isocyanate, specifically a method for preparing an isocyanate by combining a supercritical phosgenation method with a piping method.

In one aspect, the present application provides a method for preparing an isocyanate, which comprises the following steps:
(a) mixing a reactant amine stream and a phosgene stream at a temperature of −5 to 5° C., to obtain a mixture of reactant amine and phosgene;
(b) adjusting the temperature of the mixture obtained in Step (a) to 182° C. to 205° C. so that phosgene is in a supercritical state, and reacting in a supercritical reactor for at least 15 minutes;
(c) reacting the reaction product mixture obtained in Step (b) under the condition of reduced pressure, for a reaction time of no more than 30 seconds.

In some embodiments, the reduced pressure in Step (c) is carried out in a reduced pressure reactor. For example, Step (c) is introducing the reaction product mixture from the supercritical reactor of Step (b) into a reduced pressure reactor for the reaction.

In some embodiments, the method further comprises Step (d): collecting the product. In some embodiments, Step (d) comprises setting a quench zone at an outlet of the reduced pressure reactor, so that the reaction product mixture obtained in Step (c) contacts with a quench medium stream introduced in the quench zone, reducing the temperature of the reaction product mixture obtained in Step (c) to 170° C. or less.

In some embodiments, the method further comprises Step (e): purifying the product. In some embodiments, Step (e) comprises:
1) introducing the reaction product mixture obtained in Step (c) or Step (d) into a degassing tower, where hydrogen chloride and phosgene in the reaction product mixture overflow from the top of the degassing tower and enter a hydrogen chloride/phosgene separation tower, wherein the hydrogen chloride overflowing from the top of the separation tower is refined by a tail gas removal treatment unit, to form by-product hydrochloric acid;
2) recovering the phosgene from the bottom of the separation tower in Sub-step 1) for recycled use, to form the phosgene stream in Step (a);
3) collecting isocyanate and by-product in the reaction product mixture from the bottom of the degassing tower in Sub-step 1), and passing the isocyanate and by-product through a light-component removal tower, to remove light-component by-product;
4) collecting isocyanate and heavy-component by-product from the bottom of the light-component removal tower in Sub-step 3), passing the isocyanate and heavy-component by-product through a refining tower, collecting the isocyanate from the refining tower, and removing the heavy-component by-product.

In some embodiments, the light-component by-product in Sub-step 3) is selected from the group consisting of piperidine, multi-hydropyridine and a combination thereof. In some embodiments, the heavy-component by-product in Sub-step 4) is selected from the group consisting of tar, a PDI polymer, by-product urea and any combination thereof.

In some embodiments, Step (a) is carried out before Step (b) and Step (c).

In some embodiments, no organic solvents are used in each of Step (a), Step (b), and Step (c).

In some embodiments, the reactant amine stream and the phosgene stream are mixed in the supercritical reactor in Step (a).

In some embodiments, the mixed reactant amine stream and the phosgene stream form suspended particles by shear emulsification. In some embodiments, the shear emulsification is carried out in the supercritical reactor.

In some embodiments, the suspended particles have a diameter of less than or equal to 100 μm. In some embodiments, the suspended particles have a diameter of less than or equal to 50 μm. In some embodiments, the suspended particles have a diameter of less than or equal to 20 μm.

In some embodiments, the reactant amine stream and the phosgene stream pass through a homogenizing pump for shear emulsification uniformly in Step (a). In some embodiments, the uniform shear emulsification is achieved by controlling the lift, rotating speed, torque, suction and/or shear homogenization time of the homogenizing pump. In some embodiments, the circulating output volume of the homogenization pump is controlled to be greater than or equal to 10 times the volume of the liquid holdup in the supercritical reactor.

In some embodiments, the phosgene stream in Step (a) is stoichiometric excess relative to the amino groups of the reactant amine stream.

In some embodiments, the feed ratio (molar ratio) of the phosgene stream and the reactant amine stream in Step (a) is from 7:1 to 25:1. In some embodiments, the feed ratio (molar ratio) of the phosgene stream and the reactant amine stream in Step (a) is from 10:1 to 20:1. In some embodiments, the feed ratio (molar ratio) of the phosgene stream and the reactant amine stream in Step (a) is 12:1.

In some embodiments, the phosgene stream in Step (a) exists in liquid form.

In some embodiments, the reaction temperature in Step (c) is 150° C. to 450° C. In some embodiments, the reaction temperature in Step (c) is 200° C. to 400° C. In some embodiments, the reaction temperature in Step (c) is 250° C. to 350° C.

In some embodiments, the reaction pressure in Step (c) is 15 KPa to 500 KPa. In some embodiments, the reaction pressure in Step (c) is 50 KPa to 300 KPa. In some embodiments, the reaction pressure in Step (c) is 50 KPa to 110 KPa. In some embodiments, the reaction pressure in Step (c) is 80 KPa to 100 KPa.

In some embodiments, the residence time during the reaction in Step (c) is 0.5 seconds to 30 seconds. In some embodiments, the residence time during the reaction in Step (c) is 1.5 seconds to 20 seconds. In some embodiments, the residence time during the reaction in Step (c) is 2.5 seconds to 10 seconds.

In some embodiments, the product collection temperature is 170° C. or less in Step (d). In some embodiments, the product collection temperature is 80° C. to 150° C. in Step (d). In some embodiments, the product collection temperature is 110° C. to 140° C. in Step (d).

In some embodiments, in Step (d), the temperature of the reaction product mixture obtained in Step (c) is reduced rapidly by utilizing the latent heat of vaporization of the quench medium. In some embodiments, the quench medium in Step (d) is selected from the group consisting of an organic solvent, an isocyanate, phosgene, hydrogen chloride, an inert carrier gas, and any combination thereof. In some embodiments, the organic solvent is selected from the group consisting of dichloromethane, chlorobenzene, o-dichlorobenzene, benzene, toluene, xylene, hexane, tetrahydrofuran, chloronaphthalene and any combination thereof. In some embodiments, the quench medium in Step (d) is a liquid. In some embodiments, the quench medium in Step (d) is liquid phosgene.

In some embodiments, the reduced pressure reactor is a tubular reactor. In some embodiments, the inner diameter of the pipe in the reduced pressure reactor is 4 to 9 mm.

In some embodiments, the isocyanate is a diisocyanate. In some embodiments, the isocyanate is an aliphatic diisocyanate or an aromatic diisocyanate. In some embodiments, the isocyanate is selected from the group consisting of methylene diphenyl diisocyanate as a pure isomer or as a mixture of isomers, toluene diisocyanate as a pure isomer or as a mixture of isomers, 2,6-xylyl isocyanate, 1,5-naphthalene diisocyanate, methyl isocyanate, ethyl isocyanate, propyl isocyanate, isopropyl isocyanate, butyl isocyanate, isobutyl isocyanate, t-butyl isocyanate, pentyl isocyanate (e.g., pentamethylene diisocyanate), t-pentyl isocyanate, isopentyl isocyanate, neopentyl isocyanate, hexyl isocyanate (e.g., hexamethylene diisocyanate), cyclopentyl isocyanate, cyclohexyl isocyanate, and phenyl isocyanate (e.g., p-phenylene diisocyanate).

In some embodiments, the isocyanate is pentamethylene diisocyanate (PDI), hexamethylene diisocyanate (HDI), isophorone diisocyanate (IPDI) or methylcyclohexane diisocyanate (HTDI). In some embodiments, the reactant amine has a structural formula of R(NH₂)ₙ, wherein n is 1, 2 or 3, and R is an aliphatic or aromatic hydrocarbyl group. In some embodiments, n is 2, and R is an aliphatic hydrocarbyl group. In some embodiments, n is 2, and R is an aliphatic hydrocarbyl in group having 2-10 carbon atoms. In some embodiments, n is 2, and R is a linear or cyclic aliphatic hydrocarbyl group having 3-10 carbon atoms.

In some embodiments, the reactant amine exists in a free form.

In some embodiments, the reactant amine exists as an amine salt. In some embodiments, the amine salt is selected from the group consisting of a hydrochloride, a sulfate, a bisulfate, a nitrate, and a carbonate.

In some embodiments, the reactant amine is one or more selected from the group consisting of ethyl amine, butyl amine, pentamethylene diamine, hexamethylene diamine, 1,4-diamino butane, 1,8-diamino octane, aniline, p-phenylene diamine, m-xylylene diamine, toluene diamine, 1,5-naphthalene diamine, diphenylmethane diamine, dicyclohexylmethane diamine, m-cyclohexyldimethylene diamine, isophorone diamine, methyl cyclohexane diamine and trans-1,4-cyclohexane diamine.

In some embodiments, the reactant amine is selected from the group consisting of PDA, PDA hydrochloride, HDA, HDA hydrochloride, IPDA, IPDA hydrochloride, HTDA, and HTDA hydrochloride.

### BRIEF DESCRIPTION OF DRAWINGS

The above and other features of the present application will be more fully and clearly understood by the following description and appended claims combined with the drawings. It can be understood that these drawings only depict a number of embodiments of the disclosure of the present application, and therefore should not be considered as a limitation on the scope of the disclosure of the present application. The disclosure of the present application will be explained more clearly and in detail with reference to the drawings.

FIG. 1 shows a schematic flow chart of a method for preparing an isocyanate according to an embodiment of the present application; wherein: 01 is supercritical reactor, 02 is reduced pressure reactor, 03 is quencher, 04 is degassing tower, 05 is phosgene/hydrogen chloride separation tower, 06 is light-component removal tower, 07 is product refining tower.

### DETAILED DESCRIPTION OF THE INVENTION

The illustrative embodiments described in the detailed description, drawings, and claims are not intended to be limiting. Other embodiments may be adopted and other changes may be made without departing from the spirit or scope of the subject matter of the present application. It can be understood that various aspects of the disclosure of the present application generally described in the present application and graphically illustrated in the drawings can be configured, substituted, combined, and designed in a variety of different compositions, and all of which clearly constitute a part of the content of the present application.

In an aspect, the present application provides a method for preparing an isocyanate, which comprises the following steps:
(a) mixing a reactant amine stream and a phosgene stream at a temperature of −5 to 5° C., to obtain a mixture of reactant amine and phosgene;
(b) adjusting the temperature of the mixture obtained in Step (a) to 182° C. to 205° C. so that the phosgene is in a supercritical state, and reacting in a supercritical reactor for at least 15 minutes;
(c) reacting the reaction product mixture obtained in Step (b) under the condition of reduced pressure, for a reaction time of no more than 30 seconds.

As used herein, “isocyanate” refers to a class of compounds containing one or more (for example, two, three, four, five, six, seven, eight, min, ten or more) isocyanate groups (R—N═C═O), including aliphatic isocyanates, aromatic isocyanates, unsaturated isocyanates, halogenated isocyanates, thioisocyanates, phosphorus-containing isocyanates, inorganic isocyanates, and blocked isocyanates, etc. In some embodiments, the isocyanate in the present application is a diisocyanate. In some embodiments, the isocyanate in the present application is an aliphatic diisocyanate or an aromatic diisocyanate. In some embodiments, the isocyanate in the present application includes an aromatic isocyanate, or an aliphatic isocyanate. For example, the aromatic isocyanate includes methylene diphenyl diisocyanate as a pure isomer or as a mixture of isomers, toluene diisocyanate as a pure isomer or as a mixture of isomers, 2,6-xylyl isocyanate, 1,5-naphthalene diisocyanate, etc. The aliphatic isocyanate includes methyl isocyanate, ethyl isocyanate, propyl isocyanate, isopropyl isocyanate, butyl isocyanate, isobutyl isocyanate, t-butyl isocyanate, pentyl isocyanate, t-pentyl isocyanate, isopentyl isocyanate, neopentyl isocyanate, hexyl isocyanate, cyclopentyl isocyanate, cyclohexyl isocyanate, phenyl isocyanate, etc. In some embodiments, the isocyanate in the present application is selected from the group consisting of pentane diisocyanate, hexane diisocyanate, p-phenylene diisocyanate, and toluene diisocyanate. In some embodiments, the isocyanate in the present application is pentamethylene diisocyanate (PDI), hexamethylene diisocyanate (HDI), isophorone diisocyanate (IPDI), or methylcyclohexane diisocyanate (HTDI).

Step (a), Step (b), Step (c) and optional Step (d) and Step (e) of the method for preparing an isocyanate according to the present application are described in detail below.

### 1. Step (a)

In Step (a) of the present application, a reactant amine stream and a phosgene stream are mixed at a temperature of −5 to 5° C., to obtain a mixture of reactant amine and phosgene.

As used herein, the “reactant amine” refers to a compound having amino (—NH₂) group as a starting material for preparing isocyanate. In some embodiments, the reactant amine has a structural formula of R(NH₂)ₙ, wherein n is 1, 2 or 3, and R is an aliphatic or aromatic hydrocarbyl group. In some embodiments, n is 2, and R is an aliphatic hydrocarbyl group. In some embodiments, n is 2, and R is an aliphatic, alicyclic or aromatic hydrocarbyl group having 2-10 carbon atoms (for example, 2, 3, 4, 5, 6, 7, 8, 9, or 10 carbon atoms). In some embodiments, n is 2, and R is a linear or cyclic aliphatic hydrocarbyl group having 3-10 carbon atoms (for example, 3, 4, 5, 6, 7, 8, 9, or 10 carbon atoms).

In some embodiments, the reactant amine is a primary amine, that is, it has an NH₂ group. In some embodiments, the reactant amine is a diamine, that is, it has 2 NH₂ groups. In some embodiments, the reactant amine is one or more selected from the group consisting of ethyl amine, butyl amine, pentamethylene diamine, hexamethylene diamine, 1,4-diamino butane, 1,8-diamino octane, aniline, p-phenylene diamine, m-xylylene diamine, toluene diamine, 1,5-naphthalene diamine, diphenylmethane diamine, dicyclohexylmethane diamine, m-cyclohexyldimethylene diamine, isophorone diamine, methyl cyclohexane diamine, and trans-1,4-cyclohexane diamine. In some embodiments, the reactant amine is selected from the group consisting of pentamethylene diamine (e.g., 1,5-pentamethylene diamine), hexamethylene diamine (e.g., 1,6-hexamethylene diamine), p-phenylene diamine, isophorone diamine, methyl cyclohexane diamine, and toluene diamine. In some embodiments, the reactant amine is pentamethylenediamine (PDA).

In some embodiments, the reactant amine exists in a free form. The term “free” refers to an amine compound in a non-salt form. An amine compound in a free form can be different from those in various salt forms in respect of some physical and/or chemical properties, for example, in solubility in a polar solvent. The amine compound in a free form can also be the same as or similar to those in various salt forms in respect of some physical and/or chemical properties.

In some embodiments, the reactant amine exists as an amine salt. In some embodiments, the amine salt is selected from the group consisting of a hydrochloride, a sulfate, a bisulfate, a nitrate, and a carbonate.

In some embodiments, the reactant amine is one or more selected from the group consisting of pentamethylenediamine (PDA), PDA hydrochloride, hexamethylenediamine (HDA), HDA hydrochloride, isophorone diamine (IPDA), IPDA hydrochloride, methylcyclohexane diamine (HTDA) and HTDA hydrochloride.

In a conventional method for preparing an isocyanate, an organic solvent is usually used to disperse the reactant amine, or an inert carrier gas (for example, nitrogen, carbon dioxide, carbon monoxide, helium, or argon) is used to facilitate the gasification of the reactant amine and achieve a more suitable dispersion effect. However, in the present invention, the inventors unexpectedly found that neither an organic solvent nor an inert carrier gas needs to be used when mixing the reactant amine and phosgene in Step (a). In some embodiments, the reactant amine stream and the phosgene stream are mixed in a supercritical reactor (for example, the two are introduced and mixed in the supercritical reactor). In some embodiments, the reactant amine stream and the phosgene stream are mixed outside the supercritical reactor, that is, the two are mixed in other containers or pipelines before being introduced into the supercritical reactor. For ease of mixing of the reactant amine stream and the phosgene stream to facilitate the subsequent reaction, in Step (a), the reactant amine stream and the phosgene stream can be mixed and then shear emulsified to form suspended particles. In some embodiments, the suspended particles have a diameter of, for example, less than or equal to 100 μm, or less than or equal to 50 μm, or less than or equal to 20 μm. In some specific embodiments, the diameter of the suspended particles is 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35μ, 40 μm, 45μ, 50 μm, 55 μm, 60μ, 65μ, 70μ, 75 μm, 80μ, 85 μm, 90 μm, 95 μm, or 100 μm, or a range between any two of the above values. Without being limited by any theory, it is believed that the smaller the diameter of the suspended particles formed, the more conducive to the subsequent reaction of the reactant amine and phosgene.

In Step (a), the reactant amine stream and the phosgene stream can be shear emulsified by any method known in the art after being mixed. For example, the reactant amine and phosgene can be shear emulsified uniformly by mechanical shearing using a high-speed shear emulsifier, a high-gravity mixer, a homogenizing pump and the like. In some embodiments, the reactant amine stream and the phosgene stream are mixed and then shear emulsified uniformly by a homogenizing pump. In some embodiments, the shear emulsification is carried out in the supercritical reactor. The homogenizing pump used in the present application is commercially available, such as DHX homogenizing pump purchased from Ningbo Durrex Pump Industry Co., Ltd.

The homogenizing pump can be located inside the supercritical reactor (called “internal homogenizing pump” in this situation), or outside the supercritical reactor (called “external homogenizing pump” in this situation). In some embodiments of the present application, the homogenizing pump is an internal homogenizing pump. When an internal homogenizing pump is used, the uniform shear emulsification can be achieved by those skilled in the art by preferably controlling the lift, rotational speed, torque, suction and/or shear homogenization time of the homogenizing pump. Specific values can be determined according to the size of the supercritical reactor and/or the experience of those skilled in the art. In some embodiments, the rotational speed of the internal homogenizing pump is set to be 1000 to 3000 r/min (for example, 1000 r/min, 1100 r/min, 1200 r/min, 1300 r/min, 1400 r/min, 1500 r/min, 1600 r/min, 1700 r/min, 1800 r/min, 1900 r/min, 2000 r/min, 2100 r/min, 2200 r/min, 2300 r/min, 2400 r/min, 2500 r/min, 2600 r/min, 2700 r/min, 2800 r/min, 2900 r/min, 3000 r/min, or any value or range between any two of the above values). In some embodiments, the flow rate of the internal homogenizing pump is set to be 120 to 250 m³/h (for example, 120 m³/h, 130 m³/h, 140 m³/h, 150 m³/h, 160 m³/h, 170 m³/h, 180 m³/h, 190 m³/h, 200 m³/h, 210 m³/h, 220 m³/h, 230 m³/h, 240 m³/h, 250 m³/h, or any value or range between any two of the above values). In some embodiments, the pressure of the internal homogenizing pump is set to be 0.1 to 1.2 MPa (for example, 0.1 MPa, 0.2 MPa, 0.3 MPa, 0.4 MPa, 0.5 MPa, 0.6 MPa, 0.7 MPa, 0.8 MPa, 0.9 MPa, 1 MPa, 1.1 MPa, 1.2 MPa, or any value or range between any two of the above values). In some embodiments, the inlet of the internal homogenizing pump is set to be 80 mm to 110 mm (for example, 85 mm, 90 mm, 95 mm, 96 mm, 97 mm, 98 mm, 99 mm, 100 mm, 105 mm, 110 mm, or any value or range between any two of the above values). In some embodiments, the outlet of the internal homogenizing pump is set to be 60 mm to 90 mm (for example, 65 mm, 70 mm, 75 mm, 76 mm, 77 mm, 78 mm, 79 mm, 80 mm, 81 mm, 82 mm, 83 mm, 84 mm, 85 mm, 90 mm, or any value or range between any two of the above values).

Without being limited by any theory, it is believed that when the circulating output volume of the internal homogenizing pump is n times of the liquid holdup in the supercritical reactor, the internal homogenizing pump has performed at least n times of shear emulsification to the reactant amine and phosgene therein. For example, when the circulating output volume of the internal homogenizing pump is 10 times of the liquid holdup in the supercritical reactor, the internal homogenizing pump has performed 10 times of shear emulsification to the reactant amine and phosgene therein. In some embodiments, a circulating output volume of the internal homogenizing pump that is greater than or equal to 10 times of the liquid holdup in the supercritical reactor is used as a basis for determining whether uniform shear emulsification is achieved. For example, when the circulating output volume of the internal homogenizing pump is greater than or equal to 10 times of the liquid holdup in the supercritical reactor (for example, the circulating output volume of the internal homogenizing pump is 11 times, 12 times, 13 times, 14 times, 15 times, 16 times, 17 times, 18 times, 19 times, 20 times, 25 times, 30 times, 35 times, 40 times, 45 times, 50 times or more of the liquid holdup in the supercritical reactor), the mixture of the reactant amine and phosgene is determined to achieve uniform shear emulsification.

In some embodiments, the homogenizing pump is an external homogenizing pump. The homogenizing pump is located outside the supercritical reactor, and the mixture after shear emulsification is directly transported to the supercritical reactor from the outlet of the homogenizing pump. When an external homogenizing pump is used, uniform shear emulsification can be achieved by those skilled in the art by controlling the type and rotational speed of and the residence time of the material in the external homogenization pump. In some embodiments, the rotational speed of the external homogenizing pump is set to be 1000 to 3000 r/min (for example, 1000 r/min, 1100 r/min, 1200 r/min, 1300 r/min, 1400 r/min, 1500 r/min, 1600 r/min, 1700 r/min, 1800 r/min, 1900 r/min, 2000 r/min, 2100 r/min, 2200 r/min, 2300 r/min, 2400 r/min, 2500 r/min, 2600 r/min, 2700 r/min, 2800 r/min, 2900 r/min, 3000 r/min, or any value or range between any two of the above values). In some embodiments, the flow rate of the external homogenizing pump is set to be 120 to 250 m³/h (for example, 120 m³/h, 130 m³/h, 140 m³/h, 150 m³/h, 160 m³/h, 170 m³/h, 180 m³/h, 190 m³/h, 200 m³/h, 210 m³/h, 220 m³/h, 230 m³/h, 240 m³/h, 250 m³/h, or any value or range between any two of the above values). In some embodiments, the pressure of the external homogenizing pump is set to be 0.1 to 1.2 MPa (for example, 0.1 MPa, 0.2 MPa, 0.3 MPa, 0.4 MPa, 0.5 MPa, 0.6 MPa, 0.7 MPa, 0.8 MPa, 0.9 MPa, 1 MPa, 1.1 MPa, 1.2 MPa, or any value or range between any two of the above values). In some embodiments, the inlet of the external homogenizing pump is set to be 80 mm to 110 mm (for example, 85 mm, 90 mm, 95 mm, 96 mm, 97 mm, 98 mm, 99 mm, 100 mm, 105 mm, 110 mm, or any value or range between any two of the above values). In some embodiments, the outlet of the external homogenizing pump is set to be 60 mm to 90 mm (for example, 65 mm, 70 mm, 75 mm, 76 mm, 77 mm, 78 mm, 79 mm, 80 mm, 81 mm, 82 mm, 83 mm, 84 mm, 85 mm, 90 mm, or any value or range between any two of the above values).

The “supercritical reactor” in the present application refers to a reactor that can enable the reactant to be in a supercritical state or the reaction is carried out in a supercritical medium. The parameters such as pressure and temperature therein can be adjusted according to the reaction requirements (such as the species and properties of reactants, etc.) to ensure the supercritical reaction. The supercritical reactor used in the present invention can be any commercially available supercritical reactor, such as the L series high-temperature and high-pressure supercritical reactor purchased from Shanghai Labe Scientific Instrument Co., Ltd.

In Step (a), the reactant amine stream can be fed to the supercritical reactor in a single substream containing the reactant amine, or in multiple substreams (for example, 2, 3, 4, 5 or more) containing the reactant amine. Similarly, in Step (a), the phosgene stream can be fed into the supercritical reactor in a single substream containing phosgene, or in multiple substreams (for example, 2, 3, 4, 5 or more) containing phosgene. When the reactant amine stream (or phosgene stream) in Step (a) is fed into the supercritical reactor in multiple substreams containing the reactant amine (or phosgene), the multiple substreams can be fed into the supercritical reactor at the same position or at different positions.

The process of preparing the isocyanate often requires the addition of a large amount of excess phosgene, because when the concentration of phosgene is insufficient, the formed isocyanate will react with excess amine to form urea or other high-viscosity solid by-products. Therefore, to prevent the formation of by-products, it is preferable to provide excess phosgene. For example, in some embodiments, the phosgene stream in Step (a) is stoichiometric excess relative to the amino groups of the reactant amine stream. For example, the molar ratio of the phosgene to the amino groups of the reactant amine is generally 1.1:1 to 50:1 (for example, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 11:1, 15:1, 20:1, 25:1, 30:1, 35:1, 40:1, 45:1, 50:1, and a range between any two of the above values). In some embodiments, in the supercritical reactor, phosgene is used in a stoichiometrically excess amount relative to the amino groups of the reactant amine, which is 0% to 250% (for example, 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, or 250%, etc.) higher than the theoretical value. When the reactant amine stream (and/or phosgene stream) in Step (a) is fed into the supercritical reactor in multiple substreams containing the reactant amine (and/or phosgene), the total phosgene stream produced by the sum of the multiple substreams containing phosgene is stoichiometric excess relative to the amino groups of the total reactant amine stream produced by the sum of the multiple substreams containing the reactant amine.

In some embodiments, the feed ratio (molar ratio) of the phosgene stream and the reactant amine stream in Step (a) is from 7:1 to 25:1 (for example, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, or any value between any two ratios above). Preferably, the feed ratio (molar ratio) of the phosgene stream and the reactant amine stream in Step (a) is from 10:1 to 20:1.

In Step (a), the phosgene contained in the phosgene stream can be fresh phosgene or recycled phosgene. The term “fresh phosgene” refers to a stream containing phosgene that has not been recycled from a phosgenation process and has not gone through any reaction stage involving a phosgenation reaction after the synthesis of phosgene, usually from chlorine and carbon monoxide. The term “recycled phosgene” refers to a stream containing phosgene collected from a tail gas of a reaction for preparing an isocyanate by phosgenation. As mentioned above, in the process of preparing an isocyanate by a gas-phase method, excess phosgene is often required, so there will be a large amount of phosgene in the reaction tail gas, and recycling the phosgene in the tail gas can achieve the purpose of reducing the production cost. In some embodiments, the phosgene stream in Step (a) exists in liquid form.

In some embodiments, the reactant amine stream and the phosgene stream in Step (a) are mixed at a low temperature, for example, any temperature between-5 to 5° C., for example, −5° C., −4° C., −3° C., −2° C., −1° C., 0° C., 1° C., 2° C., 3° C., 4° C., 5° C., or any value between any two of the above values. The mixing can be carried out at any constant temperature between-5 and 5° C., or at varying temperatures between-5 and 5° C. In one embodiment, the mixing is carried out at a constant temperature of 0° C.

In some embodiments, Step (a) is carried out before Step (b) and Step (c). That is, the reactant amine and phosgene are mixed before reaction, and then they are heated and reacted together. One of the advantages of this operation is that it prevents the reactant amine (for example, an amine salt) from decomposition at high temperature or from self-cyclization to produce by-products.

### 2. Step (b)

In Step (b) of the present application, the temperature of the mixture obtained in Step (a) is adjusted to 182° C. to 205° C. such that phosgene is in a supercritical state, and reacted for at least 15 minutes in a supercritical reactor.

For example, pentamethylene diamine hydrochloride and phosgene are used as starting materials, and the main reaction in Step (b) is as follows:

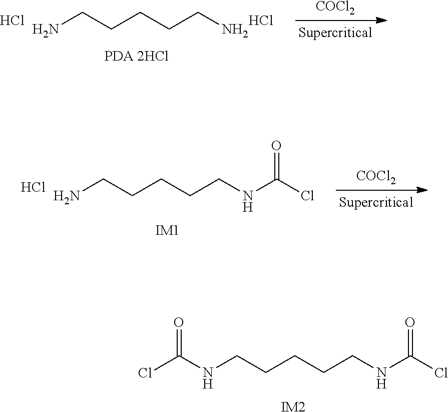

In some embodiments, in Step (b), the temperature in the supercritical reactor is adjusted to 182-205° C. so that the phosgene is in a supercritical state, and then the reactant amine is reacted with the phosgene for not less than 15 minutes.

The “supercritical state” of the present application refers to a state where the temperature and pressure of a fluid are increased to a temperature higher than the critical temperature and a pressure higher than the critical pressure, so that the fluid is in a state between gas and liquid. Many physical and chemical properties of substances in supercritical state are between gas and liquid, and have the advantages of both, for example, high solubility, good diffusivity, and easy control, etc., which have not only similar solubility and heat transfer coefficient to the liquid, but also similar viscosity and diffusion coefficient to the gas. For example, the critical temperature of phosgene is 182° C. and the critical pressure is 5.674 MPa. When the temperature of phosgene is 182° C. or above and the pressure is 5.674 MPa or above, phosgene is in supercritical state.

Generally, the amine salt is almost insoluble in any common organic solvent and can only be dispersed in the solvent. The inventors of the present application unexpectedly found that phosgene in supercritical state can dissolve the reactant amine (especially amine salt), so that the phosgene in supercritical state is reacted with the reactant amine. The phosgene in supercritical state not only acts as the solvent of the reactant amine, but also acts as the starting material of the reaction. Accordingly, no other solvents are required for dissolving the reactant amine, and the reaction rate is greatly improved. In addition, when the reactant amine is an amine salt (e.g., PDA hydrochloride), it can also effectively avoid cyclization deterioration in the heating process of the amine salt alone. In some embodiments, no organic solvent is used in Step (b) of the present application.

In some embodiments, Step (b) is carried out under supercritical phosgenation reaction conditions at a pressure of ≥5 MPa (for example, 5.1 MPa, 5.2 MPa, 5.3 MPa, 5.4 MPa, 5.5 MPa, 5.6 MPa, 5.7 MPa, 5.8 MPa, 5.9 MPa, 6 MPa, 6.1 MPa, 6.2 MPa, 6.3 MPa, 6.4 MPa, 6.5 MPa, 6.6 MPa, 6.7 MPa, 6.8 MPa, 6.9 MPa, 7 MPa, 7.5 MPa, 8 MPa, 8.1 MPa, 8.2 MPa, 8.3 MPa, 8.4 MPa, 8.5 MPa, 8.6 MPa, 8.7 MPa, 8.8 MPa, 8.9 MPa, 9 MPa, 9.1 MPa, 9.2 MPa, 9.3 MPa, 9.4 MPa, 9.5 MPa, 9.6 MPa, 9.7 MPa, 9.8 MPa, 9.9 MPa, 10 MPa, 11 MPa, 12 MPa or higher) and a temperature between 182° C. and 205° C. (for example, 182° C., 183° C., 184° C., 185° C., 186° C., 187° C., 188° C., 189° C., 190° C., 191° C., 192° C., 193° C., 194° C., 195° C., 196° C., 197° C., 198° C., 199° C., 200° C., 201° C., 202° C., 203° C., 204° C., 205° C., or any value between any two of the above values). In some embodiments, the reaction pressure is 5.2 MPa and the reaction temperature is 182° C. in Step (b). In some embodiments, the reaction pressure is 6.2 MPa and the reaction temperature is 182° C. in Step (b). In some embodiments, the reaction pressure is 6.3 MPa and the reaction temperature is 182° C. in Step (b). In some embodiments, the reaction pressure is 6.4 MPa and the reaction temperature is 182° C. in Step (b). In some embodiments, the reaction pressure is 6.5 MPa and the reaction temperature is 182° C. in Step (b). In some embodiments, the reaction pressure is 6.6 MPa and the reaction temperature is 182° C. in Step (b). In some embodiments, the reaction pressure is 6.7 MPa and the reaction temperature is 182° C. in Step (b). In some embodiments, the reaction pressure is 6.9 MPa and the reaction temperature is 182° C. in Step (b). In some embodiments, the reaction pressure is 7 MPa and the reaction temperature is 182° C. in Step (b). In some embodiments, the reaction pressure is 7.8 MPa and the reaction temperature is 182° C. in Step (b). In some embodiments, the reaction pressure is 8.4 MPa and the reaction temperature is 182° C. in Step (b).

In some embodiments, in Step (b), to ensure the adequate reaction between the reactant amine and phosgene, their reaction lasts for no less than 15 minutes, for example, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, or any value between any two of the above values. In some embodiments, the reaction between the reactant amine and phosgene in Step (b) lasts for 20 minutes. In some embodiments, the reaction between the reactant amine and phosgene in Step (b) lasts for no less than 30 minutes, for example, 30 minutes, 31 minutes, 32 minutes, 33 minutes, 34 minutes, 35 minutes, 36 minutes, 37 minutes, 38 minutes, 39 minutes, 40 minutes, or any value between any two of the above values.

### 3. Step (c)

In Step (c) of the present application, the reaction product mixture obtained in Step (b) is reacted under reduced pressure for no more than 30 seconds.

For example, pentamethylene diamine hydrochloride and phosgene are used as starting materials, and the main reaction in Step (c) is as follows:

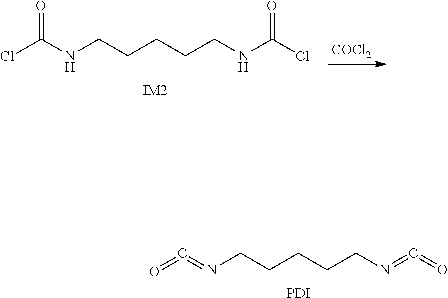

The reaction product mixture produced after Step (b) includes an acyl chloride intermediate, unreacted reactant amine and phosgene. Step (c) is a step of decomposing the acyl chloride intermediate to form an isocyanate. The present applicant unexpectedly found that the decomposition of acyl chloride under reduced pressure can significantly shorten the preparation time of isocyanate and solve the problem of high supercritical reaction pressure. In the conventional method for preparing isocyanate with phosgene, the residence time during the phosgenation reaction is several hours or even more than ten hours, and the pressure can reach 9-10 MPa. In the method of the present application, by combining the supercritical phosgenation reaction (i.e., Step (b)) with the reaction under reduced pressure (i.e., Step (c)), the preparation time of isocyanate can be shortened to less than 30 minutes, and the pressure can also be reduced to 6-8 MPa.

In some embodiments, the reaction pressure in Step (c) is 15 KPa to 500 KPa, for example, 15 KPa, 50 KPa, 60 KPa, 70 KPa, 80 KPa, 90 KPa, 100 KPa, 110 KPa, 120 KPa, 130 KPa, 140 KPa, 150 KPa, 200 KPa, 210 KPa, 220 KPa, 230 KPa, 240 KPa, 250 KPa, 260 KPa, 270 KPa, 280 KPa, 290 KPa, 300 KPa, 310 KPa, 320 KPa, 330 KPa, 340 KPa, 350 KPa, 400 KPa, 450 KPa, 500 KPa, or any value between any two of the above values. In some embodiments, the reaction pressure in Step (c) is 50 KPa to 300 KPa. In some embodiments, the reaction pressure in Step (c) is 50 KPa to 140 KPa. In some embodiments, the reaction pressure in Step (c) is 50 KPa to 110 KPa. In some embodiments, the reaction pressure in Step (c) is 70 KPa to 150 KPa.

There are many ways to achieve the reduced pressure in Step (c). In some embodiments, Step (c) is introducing the reaction product mixture from the supercritical reactor of Step (b) into a reduced pressure reactor for the reaction. For example, after Step (b) is completed, the valve between the supercritical reactor and the reduced pressure reactor is opened, so that the reaction product mixture of Step (b) is introduced into the reduced pressure reactor for reaction. In some embodiments, the reduced pressure reactor is a tubular reactor (for example, tubular reduced pressure reactor). The tubular reactor is a kind of continuous operation reactor with a tubular shape and a large length-to-diameter ratio. The length of the tubular reactor is flexible, which is characterized by achieving continuity and the reaction without back mixing. In some embodiments, the inner diameter of the pipe in the reduced pressure reactor is 4 to 9 mm (for example, 4 mm, 4.5 mm, 5 mm, 5.5 mm, 6 mm, 6.5 mm, 7 mm, 7.5 mm, 8 mm, 8.5 mm, 9 mm, or any value between any two of the above values).

In some embodiments, before introducing the reaction product mixture into the reduced pressure reactor, the reduced pressure reactor is preheated to the temperature required for the reaction in this step (i.e., the acyl chloride decomposition reaction). In some embodiments, the reaction temperature in Step (c) is 150° C. to 450° C., for example, 150° C., 160° C., 170° C., 180° C., 190° C., 200° C., 210° C., 220° C., 230° C., 240° C., 250° C., 260° C., 270° C., 280° C., 290° C., 300° C., 310° C., 320° C., 330° C., 340° C., 350° C., 360° C., 370° C., 380° C., 390° C., 400° C., 410° C., 420° C., 430° C., 440° C., 450° C., or any value between any two of the above values. Preferably, the reaction temperature in Step (c) is 200° C. to 400° C. More preferably, the reaction temperature in Step (c) is 250° C. to 350° C. In some embodiments, the reaction temperature in Step (c) is 300° C.

In some embodiments, the residence time during the reaction in Step (c) is 0.5 seconds to 30 seconds, for example, 30 seconds, 25 seconds, 20 seconds, 15 seconds, 10 seconds, 9 seconds, 8 seconds, 7 seconds, 6 seconds, 5 seconds, 4.5 seconds, 4 seconds, 3.5 seconds, 3 seconds, 2.5 seconds, 2 seconds, 1.5 seconds, 1 second, 0.5 seconds, or any value between any two of the above values. Preferably, the residence time during the reaction in Step (c) is 1.5 seconds to 20 seconds. More preferably, the residence time during the reaction in Step (c) is 2.5 seconds to 10 seconds. The residence time during the reaction in Step (c) can be controlled by various methods, for example, by controlling the flow rate of the reaction product mixture of Step (b) and/or the inner diameter of the tubular reactor.

In some embodiments, no organic solvent is used in Step (c).

### 4. Step (d)

In some embodiments, the preparation method of the present application further comprises Step (d): collecting the product.

In some embodiments, Step (d) of the present application comprises: setting a quench zone at an outlet of the reduced pressure reactor, so that the reaction product mixture obtained in Step (c) contacts with a quench medium stream introduced in the quench zone, reducing the temperature of the reaction product mixture obtained in Step (c) to 170° C. or less.

In some embodiments, the product collection temperature in Step (d) is 170° C. or less, for example, 170° C., 165° C., 160° C., 155° C., 150° C., 145° C., 140° C., 135° C., 130° C., 125° C., 120° C., 115° C., 110° C., 105° C., 100° C., 95° C., 90° C., 85° C., 80° C., 75° C., 70° C., or any value between any two of the above values. In some embodiments, the product collection temperature in Step (d) is 80° C. to 150° C. In some embodiments, the product collection temperature in Step (d) is 110° C. to 140° C.

In some embodiments, in Step (d), the temperature of the reaction product mixture obtained in Step (c) is reduced rapidly by utilizing the latent heat of vaporization of the quench medium. Organic solvents (e.g., toluene, chlorobenzene, chloronaphthalene), an isocyanate or a mixture of a solvent and an isocyanate is often used as a quench medium to reduce the temperature in the supercritical reactor in the prior art. In some embodiments, the quench medium in Step (d) is selected from the group consisting of an organic solvent, an isocyanate, phosgene, hydrogen chloride, an inert carrier gas, and any combination thereof. In some embodiments, the organic solvent is selected from the group consisting of dichloromethane, chlorobenzene, o-dichlorobenzene, benzene, toluene, xylene, hexane, tetrahydrofuran, chloronaphthalene, and any combination thereof. In some embodiments, the quench medium is liquid (e.g., liquid phosgene). In some embodiments, the temperature of the reaction product mixture obtained in Step (c) is reduced rapidly by using phosgene or a mixture of phosgene and isocyanate as a quench medium. Without being limited by any theory, it is believed that the effect of using phosgene or a mixture of phosgene and isocyanate as the quench medium is better than that of using organic solvent as the quench medium. For example, using phosgene or a mixture of phosgene and an isocyanate as the quench medium can avoid the use of organic solvent in the whole reaction system, and can also avoid the problem of inlet blockage caused by solid adhesion to the wall, so that there is no steps of solvent recovery, rectification, and recycled refining in the whole process, and the preparation process is more simple, lower energy consumption and less cost. Moreover, due to the shortened high-temperature refining process, the high-temperature residence time of the reaction product isocyanate is greatly shortened, the self-polymerization reaction is reduced, and the yield of the product is higher.

### 5. Step (e)

In some embodiments, the preparation method of the present application further comprises Step (e): purifying the product.

In some embodiments, Step (e) comprises the following sub-steps:
1) introducing the reaction product mixture obtained in Step (c) or Step (d) into a degassing tower, wherein hydrogen chloride and phosgene in the reaction product mixture overflow from the top of the degassing tower and enter a hydrogen chloride/phosgene separation tower, wherein the hydrogen chloride overflowing from the top of the separation tower is refined by a tail gas removal treatment unit, to form by-product hydrochloric acid;
2) recovering the phosgene from the bottom of the separation tower in Sub-step 1) for recycled use, to form the phosgene stream in Step (a);
3) collecting isocyanate and by-products in the reaction product mixture from the bottom of the degassing tower in Sub-step 1), and passing the isocyanate and by-products through a light-component removal tower, to remove light-component by-product;
4) collecting isocyanate and heavy-component by-product from the bottom of the light-component removal tower in Sub-step 3), passing the isocyanate and heavy-component by-product through a refining tower, collecting the isocyanate from the refining tower, and removing the heavy-component by-product.

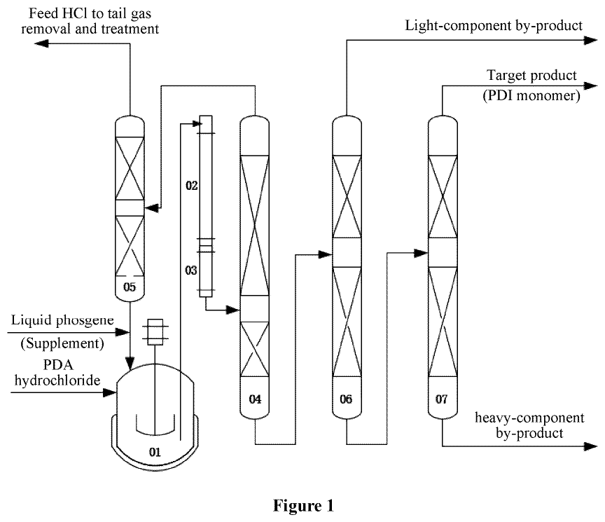

For example, as shown in FIG. 1, in Sub-step 1), the reaction product mixture obtained in Step (c) or Step (d) is introduced into a degassing tower 04, wherein hydrogen chloride and phosgene in the reaction product mixture overflow from the top of the degassing tower 04 and enter a hydrogen chloride/phosgene separation tower 05, wherein the hydrogen chloride overflowing from the top of the separation tower 05 is refined in a tail gas removal treatment unit, to form the by-product hydrochloric acid.

For example, as shown in FIG. 1, in Sub-step 2), phosgene is recovered from the bottom of the hydrogen chloride/phosgene separation tower 05, to form a phosgene stream that is fed to the supercritical reactor 01 for recycled use.

For example, as shown in FIG. 1, in Sub-step 3), the isocyanate and by-products in the reaction product mixture are collected from the bottom of the degassing tower 04, and passed through a light-component removal tower 06, to remove light-component by-product. The light-component by-product is conventional in the art. In some embodiments, the light-component by-product is selected from the group consisting of piperidine, multi-hydropyridine and a combination thereof. For example, as shown in FIG. 1, in Sub-step 4), isocyanate and heavy-component by-product are collected from the bottom of the light-component removal tower 06 and passed through a refining tower 07, the isocyanate is collected from the refining tower, and the heavy-component by-product is removed. The heavy-component by-product is conventional in the art. In some embodiments, the heavy-component by-product is selected from the group consisting of tar, a PDI self-polymer, by-product urea, and any combination thereof. When the reactant amine is amine salt, compared with a conventional gas-phase or liquid-phase phosgenation process, the step of converting the amine salt into amine is omitted in the preparation method of isocyanate in the present application; and compared with the existing salt-forming phosgenation process, the phosgene in supercritical state used in the present application can be used as not only a solvent for the reactant amine but also a starting material of the reaction, thus avoiding the use of a large amount of solvent. In addition, in the process of preparing an isocyanate using the method provided in the present application, the supercritical phosgenation reaction is combined with the reaction under reduced pressure, so that a synergistic effect is produced, for example, not only avoiding the problem of insufficient reaction rate and high pressure in the simple supercritical reaction, but also avoiding the problem of high impurity content and insufficient reaction in a simple pipe reaction. Moreover, when phosgene or a mixture of phosgene and isocyanate is used as a quench medium in the reaction process, the use of an organic solvent is avoided, and the problem of inlet blockage caused by solid adhesion to the wall can also be avoided, so that there is no steps of solvent recovery, rectification, and recycled refining in the whole process, and the preparation process is more simple, lower energy consumption and less cost.

The summary of the present application is described above, and details may be simplified, generalized, and omitted. Therefore, it is to be appreciated by those skilled in the art that this section is merely illustrative and not intended to limit the scope of the present application in any way. This summary is neither intended to define key features or essential features of the claimed subject matter, nor is it intended to be used as an aid in defining the scope of the claimed subject matter.

### EXAMPLE

To fully understand the present invention, the following examples are shown. It should be understood that these examples are provided merely for illustrative purposes and are not to be interpreted as limiting in any way.

Some abbreviations of nouns mentioned in the examples are shown in Table 1.

TABLE 1
Abbreviations of nouns

| Abbreviation | Full Name |
|---|---|
| PDI | Pentamethylene diisocyanate |
| PDA | Pentamethylene diamine |

The material ratio, reaction conditions and final yield used in the following examples are summarized in Table 2.

TABLE 2
Preparation of PDI with PDA hydrochloride (The quench medium of Example 1A-1T is
phosgene, and the quench medium of Example 1U is phosgene + PDI mixed liquid)

| | | | Inner | | | | | | |
|---|---|---|---|---|---|---|---|---|---|
| | | Molar | diameter | | Pressure | | | | |
| | | ratio of | of | | in | | | | |
| | | phosgene | reduced | | reduced | | | | |
| | | and | pressure | Reaction | pressure | Residence | | | |
| | Mixing | substrate | reactor | temperature | reactor | time | Content | Yield | |
| Example | method | (mol) | (mm) | (° C.) | (KPa) | (s) | (%) | (%) | Remark |
| 1A | PDA | 12:1 | 4 mm | 300 | 80 | 5 | 88.6 | 92.3 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1B | PDA | 12:1 | 4 mm | 300 | 80 | 5 | N/A | N/A | The raw |
| | hydrochloride | | | | | | | | materials |
| | and phosgene | | | | | | | | were |
| | were heated | | | | | | | | deteriorated |
| | separately, | | | | | | | | |
| | mixed, and | | | | | | | | |
| | sheared | | | | | | | | |
| 1C | PDA | 12:1 | 4 mm | 300 | 80 | 5 | 87.9 | 43.7 | Blockage |
| | hydrochloride | | | | | | | | occurred |
| | and liquid | | | | | | | | during the |
| | phosgene | | | | | | | | process |
| | were mixed | | | | | | | | |
| | by stirring | | | | | | | | |
| | without | | | | | | | | |
| | shearing, and | | | | | | | | |
| | heated to | | | | | | | | |
| | supercritical | | | | | | | | |
| 1D | PDA | 12:1 | 3 mm | 300 | 80 | 5 | 88.2 | 25.7 | Blockage |
| | hydrochloride | | | | | | | | occurred |
| | and liquid | | | | | | | | during the |
| | phosgene | | | | | | | | process |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1E | PDA | 12:1 | 5 mm | 300 | 80 | 5 | 82.9 | 89.4 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1F | PDA | 12:1 | 8 mm | 300 | 80 | 5 | 79.3 | 81.4 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1G | PDA | 12:1 | 10 mm | 300 | 80 | 5 | 43.4 | 40.4 | The raw |
| | hydrochloride | | | | | | | | materials |
| | and liquid | | | | | | | | were not |
| | phosgene | | | | | | | | reacted |
| | were mixed | | | | | | | | completely |
| | by stirring, | | | | | | | | |
| | sheared to | | | | | | | | |
| | uniform, and | | | | | | | | |
| | then heated to | | | | | | | | |
| | supercritical | | | | | | | | |
| 1H | PDA | 12:1 | 4 mm | 350 | 80 | 5 | 82.2 | 81.3 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1I | PDA | 12:1 | 4 mm | 400 | 80 | 5 | 63.0 | 53.2 | Many carbon |
| | hydrochloride | | | | | | | | residues and |
| | and liquid | | | | | | | | tar were |
| | phosgene | | | | | | | | present |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1J | PDA | 12:1 | 4 mm | 250 | 80 | 5 | 70.0 | 79.3 | The raw |
| | hydrochloride | | | | | | | | materials |
| | and liquid | | | | | | | | were not |
| | phosgene | | | | | | | | reacted |
| | were mixed | | | | | | | | completely |
| | by stirring, | | | | | | | | |
| | sheared to | | | | | | | | |
| | uniform, and | | | | | | | | |
| | then heated to | | | | | | | | |
| | supercritical | | | | | | | | |
| 1K | PDA | 10:1 | 4 mm | 300 | 80 | 5 | 87.7 | 88.2 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1L | PDA | 16:1 | 4 mm | 300 | 80 | 5 | 88.9 | 91.2 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1M | PDA | 20:1 | 4 mm | 300 | 80 | 5 | 89.2 | 92.5 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1N | PDA | 12:1 | 4 mm | 300 | 80 | 1 | 55.0 | 60.2 | The raw |
| | hydrochloride | | | | | | | | materials |
| | and liquid | | | | | | | | were not |
| | phosgene | | | | | | | | reacted |
| | were mixed | | | | | | | | completely |
| | by stirring, | | | | | | | | |
| | sheared to | | | | | | | | |
| | uniform, and | | | | | | | | |
| | then heated to | | | | | | | | |
| | supercritical | | | | | | | | |
| 1O | PDA | 12:1 | 4 mm | 300 | 80 | 2.5 | 69.8 | 71.2 | The raw |
| | hydrochloride | | | | | | | | materials |
| | and liquid | | | | | | | | were not |
| | phosgene | | | | | | | | reacted |
| | were mixed | | | | | | | | completely |
| | by stirring, | | | | | | | | |
| | sheared to | | | | | | | | |
| | uniform, and | | | | | | | | |
| | then heated to | | | | | | | | |
| | supercritical | | | | | | | | |
| 1P | PDA | 12:1 | 4 mm | 300 | 80 | 10 | 88.0 | 89.3 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1Q | PDA | 12:1 | 4 mm | 300 | 80 | 20 | 54.2 | 62.6 | Many carbon |
| | hydrochloride | | | | | | | | residues and |
| | and liquid | | | | | | | | tar were |
| | phosgene | | | | | | | | present |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1R | PDA | 12:1 | 4 mm | 300 | 50 | 5 | 81.9 | 85.6 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1S | PDA | 12:1 | 4 mm | 300 | 110 | 5 | 74.3 | 73.5 | |
| | hydrochloride | | | | | | | | |
| | and liquid | | | | | | | | |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1T | PDA | 12:1 | 4 mm | 300 | 140 | 5 | 68.6 | 72.3 | Many carbon |
| | hydrochloride | | | | | | | | residues and |
| | and liquid | | | | | | | | tar were |
| | phosgene | | | | | | | | present |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |
| 1U | PDA | 12:1 | 4 mm | 300 | 80 | 5 | 88.4 | 92.1 | Phosgene + |
| | hydrochloride | | | | | | | | PDI quench |
| | and liquid | | | | | | | | medium |
| | phosgene | | | | | | | | |
| | were mixed | | | | | | | | |
| | by stirring, | | | | | | | | |
| | sheared to | | | | | | | | |
| | uniform, and | | | | | | | | |
| | then heated to | | | | | | | | |
| | supercritical | | | | | | | | |

### Example 1A: Preparation of PDI with PDA Hydrochloride

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of continued the follow-up operation; while if the pressure was greater than 10 MPa, stopped the heating. When the temperature reached 182° C., the pressure was recorded as 6.6 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 Kpa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 159.4 g residue was obtained. Gas-phase quantitative analysis was performed, and the results showed that the content was 88.6% and the yield was 92.3%.

### Example 1B: Preparation of PDI with PDA Hydrochloride (PDA Hydrochloride and Phosgene were Preheated Separately without Mixing)

PDA hydrochloride and liquid phosgene were separately heated to 182° C. in two high-pressure pipes.

A tubular reactor (reduced pressure reactor) with an inner diameter of 4 mm was preheated to increase its temperature up to 300° C. The phosgene and PDA hydrochloride preheated to 182° C. were introduced into the tubular reactor at a molar ratio of 12:1. The passage time was controlled to 5 seconds, and meanwhile the pressure in the pipe was controlled to be 80 KPa. The outflow gas (liquid) was trapped by extremely cold phosgene at −20° C. to obtain a collected product liquid.

After gaseous hydrogen chloride was separated from the collected liquid, phosgene was recovered. Then the residue was subjected to gas-phase analysis, and a large number of impurities were found. The impurities were separated, analyzed and identified as multi-hydropyridine impurities.

### Example 1C: Preparation of PDI with PDA Hydrochloride (without Homogenization Shearing)

175 g (1 mol) PDA hydrochloride and 1188 g (12 mol) liquid phosgene were stirred and mixed uniformly at 0° C., and fed to a 10 L autoclave (supercritical reactor) without an internal homogenizing device.

Stirring was started. The reaction liquid was not homogenized by shearing. Then, the 10 L autoclave was heated, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.9 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 Kpa, and the outflow gas (liquid) was trapped by extremely cold phosgene at −20° C. to obtain a collected product liquid. When about half of the reaction liquid was passed, the pipe was found to be blocked and the reaction could not be continued.

After gaseous hydrogen chloride was separated from the collected liquid, phosgene was recovered, and then the residue was weighed, and 76.1 g residue was obtained. Gas-phase quantitative analysis was performed, and the results showed that the content was 87.9% and the yield was 43.7%.

### Example 1D: Preparation of PDI with PDA Hydrochloride (the Inner Diameter of the Reaction Pipe was 3 mm)

175 g (1 mol) PDA hydrochloride and 1188 g (12 mol) liquid phosgene were stirred and mixed uniformly at 0° C., and fed to a 10 L autoclave (supercritical reactor) without an internal homogenizing device.

Stirring was started. The reaction liquid was not homogenized by shearing. Then, the 10 L autoclave was heated, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 7.0 MPa, and the temperature was maintained under this condition for 20 min.

A tubular reactor (reduced pressure reactor) with an inner diameter of 3 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 Kpa, and the outflow gas (liquid) was trapped by extremely cold phosgene at −20° C. to obtain a collected product liquid. When about half of the reaction liquid was passed, the pipe was found to be blocked and the reaction could not be continued.

After gaseous hydrogen chloride was separated from the collected liquid, phosgene was recovered, and then the residue was weighed, and 44.6 g residue was obtained. Gas-phase quantitative analysis was performed, and the results showed that the content was 88.2% and the yield was 25.7%.

### Example 1E: Preparation of PDI with PDA Hydrochloride (the Inner Diameter of the Reaction Pipe was 5 mm)

175 g (1 mol) PDA hydrochloride and 1188 g (12 mol) liquid phosgene were stirred and mixed uniformly at 0° C., and fed to a 10 L autoclave (supercritical reactor) without an internal homogenizing device.

Stirring was started. The reaction liquid was not homogenized by shearing. Then, the 10 L autoclave was heated, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.5 MPa, and the temperature was maintained under this condition for 20 min.

A tubular reactor (reduced pressure reactor) with an inner diameter of 5 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 Kpa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 165.0 g residue was obtained. Gas-phase quantitative analysis was performed, and the results showed that the content was 82.9% and the yield was 89.4%.

### Example 1F: Preparation of PDI with PDA Hydrochloride (the Inner Diameter of the Reaction Pipe was 8 mm)

175 g (1 mol) PDA hydrochloride and 1188 g (12 mol) liquid phosgene were stirred and mixed uniformly at 0° C., and fed to a 10 L autoclave (supercritical reactor) without an internal homogenizing device.

Stirring was started. The reaction liquid was not homogenized by shearing. Then, the 10 L autoclave was heated, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.6 MPa, and the temperature was maintained under this condition for 20 min.

A tubular reactor (reduced pressure reactor) with an inner diameter of 8 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 Kpa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 157.1 g residue was obtained. Gas-phase quantitative analysis was performed, and the results showed that the content was 79.3% and the yield was 81.4%.

### Example 1G: Preparation of PDI with PDA Hydrochloride (the Inner Diameter of the Reaction Pipe was 10 mm)

175 g (1 mol) PDA hydrochloride and 1188 g (12 mol) liquid phosgene were stirred and mixed uniformly at 0° C., and fed to a 10 L autoclave (supercritical reactor) without an internal homogenizing device.

Stirring was started. The reaction liquid was not homogenized by shearing. Then, the 10 L autoclave was heated, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.6 MPa, and the temperature was maintained under this condition for 20 min.

A tubular reactor (reduced pressure reactor) with an inner diameter of 10 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 KPa, and the outflow gas (liquid) was trapped by extremely cold phosgene at −20° C. to obtain a collected product liquid. Many yellow-white solids presented in the collected liquid, which were detected to be the raw materials.

After gaseous hydrogen chloride was separated from the collected liquid, phosgene was recovered, and then the residue was weighed, and 142.4 g residue was obtained. Gas-phase quantitative analysis was performed, and the results showed that the content was 43.4% and the yield was 40.4%.

### Example 1H: Preparation of PDI with PDA Hydrochloride (Reaction at 350° C.)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 7.0 MPa, and the temperature was maintained under this condition for 20 min.

A tubular reactor (reduced pressure reactor) with an inner diameter of 4 mm was preheated to increase its temperature up to 350° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 Kpa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 151.3 g residue was obtained. Gas-phase quantitative analysis was performed, and the results showed that the content was 82.2% and the yield was 81.3%.

### Example 11: Preparation of PDI with PDA Hydrochloride (Reaction at 400° C.)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.7 MPa, and the temperature was maintained under this condition for 20 min.

A tubular reactor (reduced pressure reactor) with an inner diameter of 4 mm was preheated to increase its temperature up to 400° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 Kpa, and the outflow gas (liquid) was trapped by extremely cold phosgene at −20° C. to obtain a collected product liquid. The collected liquid contained many tar and carbon residues.

After gaseous hydrogen chloride was separated from the collected liquid, phosgene was recovered, and then the residue was weighed, and 129.2 g residue was obtained. Gas-phase quantitative analysis was performed, and results showed that the content was 63.0% and the yield was 53.2%.

### Example 1J: Preparation of PDI with PDA Hydrochloride (Reaction at 250° C.)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of continued the follow-up operation; while if the pressure was greater than 10 MPa, stopped the heating. When the temperature reached 182° C., the pressure was recorded as 6.4 MPa, and the temperature maintained under this condition for 20 min.

A tubular reactor (reduced pressure reactor) with an inner diameter of 4 mm was preheated to increase its temperature up to 250° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 KPa, and the outflow gas (liquid) was trapped by extremely cold phosgene at −20° C. to obtain a collected product liquid. The collected liquid was found to contain a white solid, which was analyzed and identified as the raw material PDA hydrochloride.

After gaseous hydrogen chloride was separated from the collected liquid, phosgene was recovered, and then the residue was weighed, and 173.3 g residue was obtained. Gas-phase quantitative analysis was performed, and the results showed that the content was 70.0% and the yield was 79.3%.

### Example 1K: Preparation of PDI with PDA Hydrochloride (Molar Ratio of PDA Hydrochloride:Phosgene=1:10)

175 g (1 mol) PDA hydrochloride and 990 g (10 mol) phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 5.7 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 KPa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 153.8 g residue was obtained. Gas-phase quantitative analysis was performed, and the results showed that the content was 87.7% and the yield was 88.2%.

### Example 1L: Preparation of PDI with PDA Hydrochloride (Molar Ratio of PDA Hydrochloride:Phosgene=1:16)

175 g (1 mol) PDA hydrochloride and 1584 g (16 mol) phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 7.8 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 KPa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 156.9 g residue was obtained. Gas-phase quantitative analysis was performed, and the results showed that the content was 88.9% and the yield was 91.2%.

### Example 1M: Preparation of PDI with PDA Hydrochloride (Molar Ratio of PDA Hydrochloride:Phosgene=1:20)

175 g (1 mol) PDA hydrochloride and 1977.5 g (20 mol) phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 8.4 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 KPa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 158.7 g residue was obtained. Gas-phase quantitative analysis was performed, and the results showed that the content was 89.2% and the yield was 92.5%.

### Example 1N: Preparation of PDI with PDA Hydrochloride (the Residence Time was 1 Second)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was 6.3 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 1 second (that is, the residence time was 1 second). During the process, the pressure in the pipe was controlled to be 80 KPa, and the outflow gas (liquid) was trapped by extremely cold phosgene at −20° C. to obtain a collected product liquid. The collected liquid was found to contain a white solid, which was analyzed and identified as the raw material PDA hydrochloride.

After gaseous hydrogen chloride was separated from the collected liquid, phosgene was recovered, and then the residue was weighed, and 167.5 g residue was obtained. Gas-phase quantitative analysis was performed, and the results showed that the content was 55.0% and the yield was 60.2%.

### Example 10: Preparation of PDI with PDA Hydrochloride (the Residence Time was 2.5 Seconds)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene); and observed the pressure. If the pressure was less than 10 MPa, continued the follow-up operation; while if the pressure was greater than 10 MPa, stopped the heating. When the temperature reached 182° C., the pressure was recorded as 6.2 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 2.5 seconds (that is, the residence time was 2.5 seconds). During the process, the pressure in the pipe was controlled to be 80 KPa, and the outflow gas (liquid) was trapped by extremely cold phosgene at −20° C. to obtain a collected product liquid. The collected liquid was found to contain a white solid, which was analyzed and identified as the raw material PDA hydrochloride.

After gaseous hydrogen chloride was separated from the collected liquid, phosgene was recovered, and then the residue was weighed, and 156.1 g residue was obtained. Gas-phase quantitative analysis was performed, and the results showed that the content was 69.8% and the yield was 71.2%.

### Example 1P: Preparation of PDI with PDA Hydrochloride (the Residence Time was 10 Seconds)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.5 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 10 seconds (that is, the residence time was 10 seconds). During the process, the pressure in the pipe was controlled to be 80 KPa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 155.3 g residue was obtained. Gas-phase quantitative analysis was performed, and the results showed that the content was 88.0% and the yield was 89.3%.

### Example 1Q: Preparation of PDI with PDA Hydrochloride (the Residence Time was 20 Seconds)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.4 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 20 seconds (that is, the residence time was 20 seconds). During the process, the pressure in the pipe was controlled to be 80 KPa, and the outflow gas (liquid) was trapped by extremely cold phosgene at −20° C. to obtain a collected product liquid. The collected liquid contained many tar and carbon residues.

After gaseous hydrogen chloride was separated from the collected liquid, phosgene was recovered, and then the residue was weighed, and 176.7 g residue was obtained. Gas-phase quantitative analysis was performed, and the results showed that the content was 54.2% and the yield was 62.6%.

### Example 1R: Preparation of PDI with PDA Hydrochloride (the Pressure in the Pipe was 50 KPa)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.6 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 50 KPa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 160.9 g residue was obtained. Gas-phase quantitative analysis was performed, and the results showed that the content was 81.9% and the yield was 85.6%.

### Example 1S: Preparation of PDI with PDA Hydrochloride (the Pressure in the Pipe was 110 KPa)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.6 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 110 KPa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 152.3 g residue was obtained. Gas-phase quantitative analysis was performed, and the results showed that the content was 74.3% and the yield was 73.5%.

### Example 1T: Preparation of PDI with PDA Hydrochloride (the Pressure in the Pipe was 140 KPa)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of continued the follow-up operation; while if the pressure was greater than 10 MPa, stopped the heating. When the temperature reached 182° C., the pressure was recorded as 6.6 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 140 KPa, and the outflow gas (liquid) was trapped by extremely cold phosgene at −20° C. to obtain a collected product liquid.

After gaseous hydrogen chloride was separated from the collected liquid, phosgene was recovered, and then the residue was weighed, and 162.3 g residue was obtained. Gas-phase quantitative analysis was performed, and the results showed that the content was 68.6% and the yield was 72.3%.

### Example 1U: Preparation of PDI with PDA Hydrochloride (Cryotrap of Phosgene+PDI was Used)

175 g (1 mol) PDA hydrochloride and 1188 g (12 mol) liquid phosgene were stirred and mixed uniformly at 0° C., and fed to a 10 L autoclave (supercritical reactor) with an internal homogenizing device.

The internal homogenizing pump and the stirring were started at the same time. The 10 L autoclave was heated after mixing by shearing uniformly, to slowly increase the temperature in the autoclave up to 182° C. (supercritical temperature of phosgene). The pressure was observed. If the pressure was less than 10 MPa, continued the follow-up operation; while if the pressure was greater than 10 MPa, stopped the heating. When the temperature reached 182° C., the pressure was recorded as 6.6 MPa, and the temperature was maintained under this condition for 20 min.

A tubular reactor (reduced pressure reactor) with an inner diameter of 4 mm was preheated to increase its temperature up to 300° C. Then the valve of the autoclave was slowly opened, and the outflow rate of the reaction liquid was controlled by adjusting the valve, so that the time for the reaction liquid to pass through the tubular reactor was 5 seconds (that is, the residence time was 5 seconds). During the process, the pressure in the pipe was controlled to be 80 KPa, and the outflow gas (liquid) was trapped by extremely cold phosgene+PDI (6000 g phosgene+220 g PDI in total) at −20° C. to obtain a collected product liquid.

After gaseous hydrogen chloride was separated from the collected liquid, phosgene was recovered, and then the residue was weighed, and 380.6 g residue was obtained. Gas-phase quantitative analysis was performed, and the results showed that the content was 88.4%, and the yield was 92.1% by subtracting 220 g PDI used for trapping by quenching.

What is claimed is:

1. A method for preparing an isocyanate, comprising the following steps:
(a) mixing a reactant amine stream and a phosgene stream at a temperature of −5 to 5° C., to obtain a mixture of reactant amine and phosgene;
(b) adjusting the temperature of the mixture obtained in Step (a) to 182° C. to 205° C. so that the phosgene is in a supercritical state, and reacting in a supercritical reactor for at least 15 minutes;
(c) reacting the reaction product mixture obtained in Step (b) under the condition of reduced pressure, for a reaction time of no more than 30 seconds.

2. The method according to claim 1, wherein the reduced pressure in Step (c) is carried out in a reduced pressure reactor.

3. The method according to claim 1 or 2, further comprising Step (d): collecting the product.

4. The method according to claim 3, wherein Step (d) comprises: setting a quench zone at an outlet of the reduced pressure reactor, so that the reaction product mixture obtained in Step (c) contacts with a quench medium stream introduced in the quench zone, reducing the temperature of the reaction product mixture obtained in Step (c) to 170° C. or less.

5. The method according to claim 3 or 4, further comprising Step (e): purifying the product.

6. The method according to claim 5, wherein Step (e) comprises:
1) introducing the reaction product mixture obtained in Step (c) or Step (d) into a degassing tower, wherein hydrogen chloride and phosgene in the reaction product mixture overflow from the top of the degassing tower and enter a hydrogen chloride/phosgene separation tower, wherein the hydrogen chloride overflowing from the top of the separation tower is refined by a tail gas removal treatment unit, to form by-product hydrochloric acid;
2) recovering the phosgene from the bottom of the separation tower in Sub-step 1) for recycled use, to form the phosgene stream in Step (a);
3) collecting isocyanate and by-product in the reaction product mixture from the bottom of the degassing tower in Sub-step 1), and passing the isocyanate and by-product through a light-component removal tower, to remove light-component by-product;
4) collecting isocyanate and heavy-component by-product from the bottom of the light-component removal tower in Sub-step 3), passing the isocyanate and heavy-component by-product through a refining tower, collecting the isocyanate from the refining tower, and removing the heavy-component by-product.

7. The method according to any one of the preceding claims, wherein Step (a) is carried out before Step (b) and Step (c).

8. The method according to any one of the preceding claims, wherein no organic solvents are used in each of the Step (a), Step (b), and Step (c).

9. The method according to any one of the preceding claims, wherein in Step (a), the reactant amine stream and the phosgene stream are introduced and mixed in the supercritical fluid reactor.

10. The method according to any one of the preceding claims, wherein in Step (a), the mixed reactant amine stream and the phosgene stream form suspended particles by shear emulsification.

11. The method according to claim 10, wherein the shear emulsification is carried out in the supercritical fluid reactor.

12. The method according to claim 10 or 11, wherein the suspended particles have a diameter of less than or equal to 100 μm, preferably less than or equal to 50 μm, more preferably less than or equal to 20 μm.

13. The method according to any one of the preceding claims, wherein in Step (a), the reactant amine stream and the phosgene stream pass through a homogenization pump for shear emulsification uniformly.

14. The method according to claim 13, wherein the shear emulsification is achieved by controlling the lift, rotating speed, torque, suction and/or shear homogenization time of the homogenizing pump.

15. The method according to claim 14, wherein the circulating output volume of the homogenization pump is controlled to be greater than or equal to 10 times the volume of the liquid holdup in the supercritical reactor.

16. The method according to any one of the preceding claims, wherein the phosgene stream in Step (a) is stoichiometric excess relative to the amino groups of the reactant amine stream.

17. The method according to any one of the preceding claims, wherein the feed ratio (molar ratio) of the phosgene stream and the reactant amine stream in Step (a) is from 7:1 to 25:1 (preferably 12:1).

18. The method according to any one of the preceding claims, wherein the phosgene stream in Step (a) exists in liquid form.

19. The method according to any one of the preceding claims, wherein the reaction temperature in Step (c) is 150° C. to 450° C., preferably 200° C. to 400° C., more preferably 250° C. to 350° C.

20. The method according to any one of the preceding claims, wherein the reaction pressure in Step (c) is 15 KPa to 500 KPa, preferably 50 KPa to 300 KPa, more preferably 50 KPa to 110 KPa.

21. The method according to any one of the preceding claims, wherein the residence time during the reaction in Step (c) is 0.5 seconds to 30 seconds, preferably 1.5 seconds to 20 seconds, more preferably 2.5 seconds to 10 seconds.

22. The method according to any one of claims 2 to 21, wherein the product collection temperature in Step (d) is 170° C. or less, preferably 80° C. to 150° C., more preferably 110° C. to 140° C.

23. The method according to any one of claims 3 to 22, wherein in Step (d), the temperature of the reaction product mixture obtained in Step (c) is reduced rapidly by utilizing the latent heat of vaporization of the quench medium.

24. The method according to any one of claims 3 to 23, wherein the quench medium in Step (d) is selected from the group consisting of an organic solvent, an isocyanate, phosgene, hydrogen chloride, an inert carrier gas, and any combination thereof.

25. The method according to claim 24, wherein the organic solvent is selected from the group consisting of dichloromethane, chlorobenzene, o-dichlorobenzene, benzene, toluene, xylene, hexane, tetrahydrofuran, chloronaphthalene, and any combination thereof.

26. The method according to any one of claims 3 to 25, wherein the quench medium in Step (d) is a liquid.

27. The method according to claim 26, wherein the quench medium in Step (d) is liquid phosgene.

28. The method according to any one of the preceding claims, wherein the reduced pressure reactor is a tubular reactor.

29. The method according to any one of the preceding claims, wherein the inner diameter of the pipe in the reduced pressure reactor is 4 to 9 mm.

30. The method according to any one of the preceding claims, wherein the isocyanate is a diisocyanate.

31. The method according to any one of the preceding claims, wherein the isocyanate is an aliphatic diisocyanate or an aromatic diisocyanate.

32. The method according to any one of the preceding claims, wherein the isocyanate is selected from the group consisting of methylene diphenyl diisocyanate as a pure isomer or as a mixture of isomers, toluene diisocyanate as a pure isomer or as a mixture of isomers, 2,6-xylyl isocyanate, 1,5-naphthalene diisocyanate, methyl isocyanate, ethyl isocyanate, propyl isocyanate, isopropyl isocyanate, butyl isocyanate, isobutyl isocyanate, t-butyl isocyanate, pentyl isocyanate (e.g., pentamethylene diisocyanate), t-pentyl isocyanate, isopentyl isocyanate, neopentyl isocyanate, hexyl isocyanate (e.g., hexamethylene diisocyanate), cyclopentyl isocyanate, cyclohexyl isocyanate, and phenyl isocyanate (e.g., p-phenylene diisocyanate).

33. The method according to any one of the preceding claims, wherein the isocyanate is PDI, HDI, IPDI or HTDI.

34. The method according to any one of the preceding claims, wherein the reactant amine has a structural formula of R(NH₂)ₙ, wherein n is 1, 2 or 3, and R is an aliphatic or aromatic hydrocarbyl group.

35. The method according to claim 34, wherein n is 2, and R is an aliphatic hydrocarbyl group.

36. The method according to claim 35, wherein n is 2, and R is an aliphatic hydrocarbyl group having 2-10 carbon atoms.

37. The method according to claim 36, wherein n is 2, and R is a linear or cyclic aliphatic hydrocarbyl group having 3-10 carbon atoms.

38. The method according to any one of the preceding claims, wherein the reactant amine exists in a free form.

39. The method according to any one of the preceding claims, wherein the reactant amine exists as an amine salt.

40. The method according to claim 39, wherein the amine salt is selected from the group consisting of a hydrochloride, a sulfate, a bisulfate, a nitrate, and a carbonate.

41. The method according to any one of the preceding claims, wherein the reactant amine is one or more selected from the group consisting of ethyl amine, butyl amine, pentamethylene diamine, hexamethylene diamine, 1,4-diamino butane, 1,8-diamino octane, aniline, p-phenylene diamine, m-xylylene diamine, toluene diamine, 1,5-naphthalene diamine, diphenylmethane diamine, dicyclohexylmethane diamine, m-cyclohexyldimethylene diamine, isophorone diamine, methyl cyclohexane diamine, and trans-1,4-cyclohexane diamine.

42. The method according to any one of the preceding claims, wherein the reactant amine is selected from the group consisting of PDA, PDA hydrochloride, HDA, HDA hydrochloride, IPDA, IPDA hydrochloride, HTDA, and HTDA hydrochloride.
